(19) United States
(12) Patent Application Publication
(10) Pub. No.: US 2023/0301765 A1
(43) Pub. Date: Sep. 28, 2023

(54) PREDICTION APPARATUS, PREDICTION SYSTEM AND PREDICTION METHOD

(71) Applicant: Anicom Holdings, Inc., Tokyo (JP)

(72) Inventors: Kazuteru KATO, Tokyo (JP); Kota WAKANA, Tokyo (JP); Ryo KIKUCHI, Tokyo (JP); Mari AKASAKA, Tokyo (JP); Rei SUGITA, Tokyo (JP); Akihito KOIZUMI, Tokyo (JP); Hiroyuki KONO, Tokyo (JP)

(73) Assignee: Anicom Holdings, Inc., Tokyo (JP)

(21) Appl. No.: 18/033,433

(22) PCT Filed: Aug. 26, 2022
(86) PCT No.: PCT/JP2022/032180

§ 371 Date: Apr. 24, 2023

(30) Foreign Application Priority Data
Aug. 31, 2021 (JP) 2021-141675

(51) Int. Cl. A61D 99/00; A01K 29/00

(52) U.S. Cl. CPC A61D 99/00; A01K 29/005; A61B 5/7267

(57) ABSTRACT

There are provided a prediction apparatus, a prediction system, and a prediction method, which predict, by an easy method, whether there is a possibility of an animal contracting a disease in a near future. The prediction apparatus includes a first prediction means that predicts the occurrence of a disease or disease-prone state in the future based on inborn data including one or more selected from the group consisting of genetic information, pedigree information, and appearance information of an animal; and a second prediction means that modifies a prediction generated by the first prediction means based on acquired data including one or more selected from the group consisting of information relating to eating habits, information relating to intestinal bacterial flora, information relating to a body, information relating to a living environment, information relating to diagnosis, a medical checkup, and an examination, information relating to a contracted disease, and information relating to medical treatment, with respect to the animal.

### TECHNICAL FIELD

The present invention relates to a prediction apparatus, a prediction system, and a prediction method, and more particularly to a prediction apparatus, a prediction system, and a prediction method, which predict, from inborn data and acquired data of an animal excluding humans, the occurrence of a disease or disease-prone state of the animal in the future.

### BACKGROUND ART

Pet animals including dogs, cats, and rabbits, among others, and domestic animals including cows and pigs, among others, are precious beings to humans. In recent years, while the average life span of animals reared by humans has remarkably increased, more and more animals contract some diseases in their lifetimes, leading to a problem of an increase in medical expenses borne by rearers.

In order to maintain the health of an animal, the physical condition management through daily diet, exercise, and the like, and the quick dealing with a poor condition, are important. However, since the animal cannot appeal the poor condition of the body by its own words, the reality is that owner of the animal becomes aware of the contraction of a disease of the animal only when the symptom has progressed and some sign, which is externally observable, has occurred. In the worst case, the animal may suddenly die without the owner becoming aware of the warning signs of the disease.

If it is understood that there is a strong possibility of an animal contracting a disease, such measures as improvements in eating habits and lifestyle, a thorough examination, and medical treatment can be taken to avoid the contraction of the disease and the worst result of death. In addition, even if not resulting in a disease, if the possibility of leading to a health condition that may result in a disease, such as obesity, high blood pressure, or hyperglycemia, is understood in advance, some measures can be taken.

Thus, there is a demand for means that recognizes, by an easy method, whether there is a possibility of an animal contracting a disease in the future, or there is a possibility of leading to a state that results in a disease.

Patent Document 1 discloses an information processing apparatus that acquires breed information representing a breed of an examination object that is an animal, and disease state information relating to the state of a disease of the examination object; predicts a disease or external wound that the examination object suffers from, based on the acquired breed information and disease state information; and extracts an animal hospital that can treat the disease or external wound of the examination object, based on the result of the prediction.

Patent Document 2 discloses a pet diagnosis guidance method including a sampling step of sampling a gene of a pet as a sample; an analysis step of analyzing the sample and discovering a mutation of the gene, which is directly connected to a disease; a specifying step of specifying, based on the mutation, a disease that is predicted to manifest in the pet; and a notification step of notifying an owner of the pet of the mutation discovered in the analysis step and the disease specified by the specifying step.

Patent Document 3 discloses a disease prediction system that predicts, from a facial image of an animal, whether the animal will contract a disease in the future.

### RELATED ART DOCUMENTS

### Patent Documents

[Patent Document 1] JP2021-82087A
[Patent Document 2] JP2020-171207A
[Patent Document 3] JP2018-19611A

### SUMMARY OF THE INVENTION

### Problems to be Solved by the Invention

None of the above related art documents, however, discloses a prediction apparatus or a prediction system, which includes a function of predicting the possibility of contraction of a disease, or the like, by taking into account a prediction using acquired data, in addition to a prediction result based on inborn data of an animal.

Thus, the objective of the present invention is to provide a prediction apparatus and a prediction method, which predict, by an easy method, whether there is a possibility of an animal contracting a disease in the future.

### Means for Solving the Problems

The present inventors have data on many animals that have pet insurance including information on their genes, pedigree, appearance, etc., and information on their intestinal bacterial flora, body weight, eating habits, living environment, etc., and, in addition to these data, they have data on whether the animals have used the insurance, that is, whether the animals have contracted diseases. The inventors found that the above-described problem could be solved by analyzing these data on animals, classifying the data into inborn data and acquired data, and modifying prediction results such as the contraction of diseases predicted based on the inborn data using the basis of the acquired data, and thus completed the present invention.

Specifically, the present invention is the following [1] to [13].

[1] A prediction apparatus including a first prediction means for predicting the occurrence of a future disease or disease-prone state in an animal based on inborn data including one or more selected from a group consisting of genetic, pedigree, and appearance information of the animal; and a second prediction means for modifying a prediction generated by the first prediction means based on acquired data including one or more selected from the group consisting of information on diet, intestinal bacterial flora, body, living environment, diagnosis, medical checkup, examination, contracted disease, and medical treatment of the animal.
[2] The prediction apparatus according to [1], further including a proposing means for proposing a preventive plan for preventing the occurrence of the disease or disease-prone state in accordance with the prediction modified by the second prediction means.
[3] The prediction apparatus according to [2], wherein the preventive plan includes a proposal for one or more changes consisting of food, exercise habits, lifestyle, living environment, clothing, and primary care doctor.
[4] The prediction apparatus according to any one of [1] to [3], wherein the modification by the second prediction means is a modification that delays the timing of occurrence or lowers the probability of occurrence of the future disease or disease-prone state predicted by the first prediction means.
[5] The prediction apparatus according to any one of [1] to [4], wherein the disease-prone state is one or more selected from the group consisting of obesity, low body weight, high blood pressure, low blood pressure, hyperglycemia, and hypoglycemia.
[6] The prediction apparatus according to any one of [1] to [5], further including a medical expense calculation means for calculating a medical expense that an owner of a pet possibly bears in the future in accordance with the prediction modified by the second prediction means.
[7] The prediction apparatus according to any one of [1] to [6], wherein the disease is a hereditary disease.
[8] The prediction apparatus according to any one of [1] to [6], wherein the disease is a lifestyle-related disease.
[9] The prediction apparatus according to any one of [1] to [8], wherein the genetic information is information on a sequence or mutation of one or more selected from the group consisting of a cancer-related gene, a progressive retinal atrophy (PRA)-related gene, a hereditary cataract-related gene, a collie eye anomaly (CEA)-related gene, a von Willebrand's disease (vWD)-related gene, an MDR1 gene, a copper-storage hepatopathy-related gene, a cystinuria-related gene, an osteogenesis imperfecta-related gene, an X-chromosome-linked muscular dystrophy-related gene, a voltage-gated chloride channel gene, an orexin-related gene, a severe combined immunodeficiency-related gene, a leukocyte adhesion deficiency (CLAD)-related gene, a cyclic neutropenia (gray collie syndrome)-related gene, a phosphofructokinase deficiency-related gene, a pyruvate kinase deficiency-related gene, and a lysosomal storage disease-related gene.
   [10] The prediction apparatus according to any one of [1] to [9], wherein the second prediction means is capable of further modifying the modified prediction by the first prediction means using acquired data other than the acquired data once used.
   [11] The prediction apparatus according to [1] to [10], further including a request reflection means for modifying the preventive plan proposed by the proposing means in accordance with a request of an owner of a pet.
   [12] A prediction system wherein the prediction apparatus according to any one of [1] to [11] and a terminal used by an owner of an animal are connected via a network.
   [13] A prediction method including a step of receiving inborn data including one or more selected from the group consisting of genetic, pedigree, and appearance information of an animal; a first prediction step of predicting the occurrence of a future disease or disease-prone state in the animal based on the inborn data; a step of obtaining acquired data including one or more selected from the group consisting of information on diet, intestinal bacterial flora, body, living environment, diagnosis/medical checkup/examination, contracted diseases, and medical treatment of the animal; and a second prediction step of modifying a prediction on the occurrence of the future disease or disease-prone state predicted from the inborn data based on the acquired data.

### Effects of the Invention

According to the present invention, there can be provided a prediction apparatus, a prediction system, and a prediction method, which predict, by an easy method, whether there is a possibility of an animal contracting a disease in the future.

### BRIEF DESCRIPTION OF THE DRAWINGS

FIG. 1 is a block diagram illustrating an embodiment of a prediction system of the present invention.

FIG. 2 is a block diagram illustrating an embodiment of a prediction apparatus of the present invention.

FIG. 3 is a block diagram illustrating an embodiment of the prediction apparatus of the present invention.

FIG. 4 is a flowchart illustrating an example of a flow of a prediction method by the prediction apparatus of the present invention.

FIG. 5 is a schematic view illustrating an example of the flow of the prediction method by the prediction apparatus of the present invention.

### MODE FOR CARRYING OUT THE INVENTION

### <Prediction Apparatus>

A prediction apparatus of the present invention includes a first prediction means that predicts the occurrence of a future disease or disease-prone state in an animal based on inborn data including one or more selected from the group consisting of information on the genetic, pedigree, and appearance of the animal; and a second prediction means that modifies a prediction generated by the first prediction means based on acquired data including one or more selected from the group consisting of information on diet, intestinal bacterial flora, body, living environment, diagnosis/medical checkup/examination, contracted diseases, and medical treatment of the animal. The animal is preferably a pet or pet animal, and more preferably, a dog, a cat, a ferret, or a rabbit.

### [First Prediction Means]

The first prediction means is a means that predicts when (time, frequency) and what (type) of disease or disease-prone state an animal with specific inborn data will develop. The method of prediction is not particularly limited. For example, using a preset program, a processor predicts, based on inborn data of an animal, whether the animal will contract a disease or enter a disease-prone state within a predetermined period. In the paragraphs below, predictions obtained by the first prediction means are sometimes referred to as “first prediction”.

As regards the time to be predicted, it is preferable not to be a short-term prediction such as within three months or a half year, but a long-term prediction such as within one year, three years, five years, or the lifetime of the animal. As regards the number of times to be predicted, it is preferable to include not only a one-time prediction, but also a multiple-time prediction or a chronical disease prediction. As regards the type to be predicted, it is preferable to be a disease or disease-prone state that occurs at a statistically significant rate based on the inborn data.

The first prediction means of the present invention may be configured to predict using models, singly or in combination, prepared in advance for each single inborn data or for each inborn data set composed of a plurality of inborn data. For example, when model 1 states that “animals with gene information 1 have disease 1 at a rate of 90% at age 5” and model 2 states that “animals with pedigree 2 have chronic disease-prone state 2 at a rate of 30% at age 10 or older”, if animal A has gene information 1 and pedigree 2, the prediction would be that “animal A contracts disease 1 at a rate of 90% at age 5 and develops disease-prone state 2 at a rate of 30% at age 10 or older”.

In addition, when the time and type of disease overlap, the configuration may be such that they are integrated for prediction. For example, when model 3-1 states that “animals with gene information 3 develop disease 3 at a rate of 30% at age 3 or older” and model 3-2 states that “animals with pedigree 3 develop disease 3 at a rate of 80% at age 7 or older”, if animal B has gene information 3 and pedigree 3, the prediction would be that “animal B contracts disease 3 at a rate of 30% at age 3 or older and the rate of developing disease 3 increases to 80% at age 7 or older”.

The first prediction means of the present invention may be configured to predict using a trained model. As this trained model, a trained model is preferable which learned a relation between inborn data and information on whether the animal contracted a disease or developed a disease-prone state such as obesity within a predetermined period. Furthermore, as the trained model, a trained model is preferable which was trained using, as training data, inborn data including one or more selected from a group consisting of genetic, pedigree, and appearance information of an animal and information on whether the animal contracted a disease or developed a disease-prone state within a predetermined period. As for the “predetermined period” in the information on whether the animal contracted a disease within a predetermined period, which is used as the training data, is preferably within three years, more preferably within two years, and still more preferably within one year.

As the trained model, artificial intelligence (AI) is preferable. Artificial intelligence (AI) is software or a system in which intelligent work performed by the human brain is simulated by a computer, and is, concretely, a computer program or the like, which comprehends a natural language used by humans, performs logical inference, or performs learning from experience. The artificial intelligence may be a general-purpose one or a purpose-specific one, or may be a deep neural network or a convolutional neural network, and publicized software can be used.

In order to generate a trained model, learning is performed for artificial intelligence using training data. The learning may be either machine learning or deep learning, and machine learning is preferable. Deep learning is a development of machine learning and is characterized by automatically finding feature quantity.

The learning method for generating a trained model is not particularly limited, and publicized software can be used. For example, DIGITS (the Deep Learning GPU Training System) publicized by NVIDIA can be used. Besides, learning may be performed by a publicly known support vector machine method publicized in, for example, “Support Vector Machine Nyumon” (“Introduction to Support Vector Machines”), Kyoritsu Shuppan, and other publications.

As machine learning, either unsupervised learning or supervised learning may be used, and supervised learning is preferable. The method of supervised learning is not particularly limited, and examples thereof include a decision tree, ensemble learning, and gradient boosting. Examples of the algorithm of machine learning, which are made public, include XGBoost, CatBoost, and LightGBM.

The information as to whether an animal contracted a disease, which is training data for learning, can be replaced with a dummy variable. The information as to whether the animal contracted a disease or entered a disease-prone state within a predetermined period can be obtained from, for example, an animal hospital or the owner of the insured animal in connection with the fact (also referred to as “accident”) of an insurance claim.

As the trained model, use may be made of a multi-modal trained model, for example, a model trained using, as training data, a plurality of pieces of information, among pieces of information selected from the group consisting of genetic, pedigree, and appearance information of the animal. In addition, the first prediction means may include a plurality of trained models. For example, the first prediction means may be configured to include a trained model trained using the animal's genetic information, a trained model trained using the animal's pedigree information, and a trained model trained using the animal's appearances. In the case of using a plurality of trained models, use may be made of a configuration in which a prediction result is computed by a majority decision of the trained models or a configuration in which a prediction result is computed by integrating predictions of the trained models.

### [Inborn Data]

The inborn data in the present invention is data including one or more selected from the group consisting of genetic, pedigree, and appearance information of an animal.

[Genetic Information]

The genetic information of the animal is information relating to a gene sequence of the animal, and examples thereof include information relating to a genome sequence, a sequence of a specific gene, SNP (Single Nucleotide Polymorphism), polymorphism, and a genetic mutation. The genetic information can be obtained by, for example, publicly known methods such as a sequencer and a gene test kit. As the genetic information, a gene sequence or a base sequence, which is known to relate to a disease or a disease-prone state such as obesity, is preferable.

Examples of genetic diseases (hereditary diseases) in an animal, for example, a dog, include cancer, progressive retinal atrophy (PRA), hereditary cataract, collie eye anomaly (CEA), von Willebrand's disease (vWD)-related gene, ivermectin sensitivity (MDR1 gene), copper-storage hepatopathy, cystinuria, osteogenesis imperfecta, X-chromosome-linked muscular dystrophy, congenital myotonia (mutation of a voltage-gated chloride channel gene), narcolepsy (mutation of an orexin-related gene), severe combined immunodeficiency, leukocyte adhesion deficiency (CLAD), cyclic neutropenia (gray collie syndrome), phosphofructokinase deficiency, pyruvate kinase deficiency, and lysosomal storage disease.

Examples of hereditary diseases in cats include dyschondroplasia, polycystic kidney, hypertrophic cardiomyopathy, glycogen storage disease (glycogenosis), pyruvate kinase deficiency, progressive retinal atrophy, and spinal muscle atrophy.

As the genetic information, sequence information of genes relating to the onset of these diseases is preferable. The gene relating to a disease is a gene in which an animal is more likely or less likely to develop a particular disease due to a mutation in a specific gene or due to a specific gene sequence.

### [Pedigree Information]

The pedigree information of an animal is information relating to a pedigree of an animal and may include, for example, a breed, a family, an ancestor, and a descendant. As the pedigree information, it is preferable that a relation to a disease and a disease-prone state is known. Cases of the disease and disease-prone state are the same as in the case of the gene information, and, as the pedigree information, the information of the pedigree relating to the onset of these diseases is preferable. The pedigree information relating to a disease is a pedigree in which an animal is more likely or less likely to develop a disease if it belongs to a specific pedigree.

### [Appearance Information]

The appearance information of an animal is an external appearance of the animal. The appearance information reflects the genetic information and pedigree and is one type of inborn element of an animal. The appearance information is preferable if it indicates a relation to a disease or a disease-prone state. Cases of the disease and disease-prone state are the same as in the case of the gene information, and, as the appearance information, the information of the appearance relating to the onset of these diseases is preferable. The appearance information relating to a disease is an appearance in which an animal is more likely or less likely to develop a disease when its appearance corresponds to a specific appearance (for example, “coat color”).

An example of data relating to appearance information is an image of the face of an animal. The format of the image is not particularly limited and may be a still image or a moving image. Trimming of an animal involves the whole body, and the part of the animal appearing in the image is not particularly limited. However, the image of the animal is preferably an image in which the face of the animal appears, more preferably a photograph taken by photographing the face of the animal from the front, and still more preferably a photograph in which the face of the animal appears in a large size. Besides, an image of the face showing up to the ears of the animal is particularly preferable to an image trimmed to show only the vicinity of the muzzle or an image trimmed to show only the vicinity of the eyes. An example of such a photograph is a photograph like a photograph of a human on a driver's license. An image used on a health insurance card of an animal is also preferable. The image may be black-and-white, gray-scale, or color. Unpreferable images are an image in which the entire face of an animal does not appear, an image with a shape being edited by image editing software, an image in which a plurality of animals appears, an image in which the face appears so small that the eyes and ears cannot be distinguished, or an unclear image. As regards the image, it is preferable that the image is subjected to normalization, and the resolution is standardized.

### [Diseases]

Diseases that are prediction objects in the present invention are not particularly limited. Preferably, the diseases are such diseases that the inborn properties such as the heredity, pedigree and appearance and the onset risk are linked, or such diseases that the onset risk is expected to be lowered, or the onset can be suppressed, by the improvement of the lifestyle or the like.

Examples of diseases, in which the inborn properties such as heredity, pedigree, and appearance and the onset risk are linked, include, in regard to dogs, progressive retinal atrophy (PRA), hereditary cataract, collie eye anomaly (CEA), von Willebrand's disease (vWD), MDR1, copper-storage hepatopathy, cystinuria, osteogenesis imperfecta, X-chromosome-linked muscular dystrophy, a voltage-gated chloride channel, orexin, severe combined immunodeficiency, leukocyte adhesion deficiency (CLAD), cyclic neutropenia (gray collie syndrome), phosphofructokinase deficiency, pyruvate kinase deficiency, and lysosomal storage disease, and include, in regard to cats, dyschondroplasia, polycystic kidney, hypertrophic cardiomyopathy, glycogen storage disease (glycogenosis), pyruvate kinase deficiency, mucopolysaccharidosis, progressive retinal atrophy, and spinal muscle atrophy.

Examples of diseases, in which the onset risk is expected to be lowered, or the onset can be suppressed, by the improvement of the lifestyle or the like, include, in regard to dogs, external otitis, dermatitis, gastroenteritis, cystitis, biliary sludge, arthritis, intervertebral disk herniation, pyoderma, diabetes, kidney failure, and cancer, and include, in regard to cats, dermatitis, conjunctivitis, urolithiasis, tumor diseases, cardiomyopathy, hyperthyroidism, feline asthma, diabetes, kidney failure, and cancer.

### [Disease-Prone State]

The disease-prone state refers to a physiological state in which the possibility of contracting a disease is high, and examples thereof are an increase in weight, a decrease in weight, lack of sleep, lack of exercise, lack of calcium, lack of vitamin, malnutrition, chronic fatigue, obesity, low body weight, high blood pressure, low blood pressure, hyperglycemia, and hypoglycemia, and are preferably obesity, low body weight, high blood pressure, low blood pressure, hyperglycemia, and hypoglycemia.

### [Second Prediction Means]

The second prediction means is means that modifies the first prediction based on acquired data of the animal. The method of modifying the prediction is not particularly limited. For example, a processor modifies the prediction as to whether the animal will contract a disease or enter a disease-prone state within a predetermined period based on acquired data of the animal using a preset program.

The second prediction means preferably modifies the prediction relating to the time of contraction of a disease and the time of entering a disease-prone state. For example, if the first prediction showed a 50% possibility of an animal contracting a kidney disease within three years, the second prediction means could defer the prediction time of the onset of the kidney disease to within five years instead of within three years by taking into account the information of eating habits of the animal.

The second prediction means preferably modifies the predictive numerical values relating to the probability of contraction of a disease and the probability of entering a disease-prone state. For example, if the first prediction showed a 50% possibility of an animal contracting a kidney disease within three years, the second prediction means could modify the possibility of contracting a kidney disease within three years to 20% by taking into account the information of the eating habits of the animal. These are examples of modification in which, based on the acquired data, the time of contraction of a disease in the first prediction is shifted to a later time, or the probability of contraction is lowered. Conversely, by reflecting on the acquired data, it is also possible to make such modifications as shifting the time of contraction of the disease in the first prediction to an earlier time or increasing the probability of contraction.

The second prediction means preferably modifies the prediction relating to whether an animal will contract a new disease or enter a disease-prone state. For example, in the case where the first prediction did not assume the contraction of diabetes in an animal, the second prediction means could modify the prediction, based on the acquired data, to say that the animal has a 50% possibility of contracting diabetes within one year.

It is preferable that the second prediction means can further modify the modified first prediction using other acquired data that is different from the acquired data once used. For example, after the possibility of disease contraction predicted by the first prediction means is modified using the information relating to intestinal bacterial flora, the prediction can further be modified using acquired data obtained thereafter such as the improvement of eating habits and the administration of a preventive drug. By repeating the modification, the prediction can be modified in real time with higher precision in accordance with events occurring in the lifetime of the animal.

In addition, it is preferable that the second prediction means modifies not only a short-term prediction, such as within three months or a half year, but also a long-term prediction, for example, a prediction as to what the possibility of disease contraction is within one year, three years, five years, or in the lifetime of the animal. It is preferable to modify not only the prediction of contraction of one type of disease but also the prediction of contraction of multiple types of diseases. An example of such a prediction is that there is a 30% possibility of contracting cancer and a 50% possibility of contracting a kidney disease within the next several years.

The second prediction means may be configured to modify the first prediction using models, singly or in combination, prepared in advance for each single acquired data or for each acquired data set composed of a plurality of acquired data.

For example, when there is a model 4 in which “in the case where the intestinal bacterial flora corresponds to a state 4, a chronic disease 4 is contracted at a rate of 50% one year later”, and when an animal C of six years old corresponds to the state 4 and the first prediction of the animal C is “the disease 4 is contracted at a rate of 80% at 10 years old”, the first prediction is modified to a prediction that “the rate of contraction of the disease 4 by the animal C is 50% one year later (seven years old) and increases to 80% at 10 years old”.

The second prediction means of the present invention, like the first prediction means, may be configured to predict using a trained model. As this trained model, a trained model is preferable that has learned the relationship between acquired data of an animal and information on whether the animal contracted a disease or entered a disease-prone state, such as obesity, within a predetermined period. Furthermore, as the trained model, a trained model is preferable which was trained using, as training data, acquired data and information on whether the animal contracted a disease or entered a disease-prone state within a predetermined period. As for the predetermined period for the information on whether the animal contracted a disease within a predetermined period used for such training data, three years or less is preferred, two years is more preferred, and one year or less is even more preferred.

When the second prediction means uses such a trained model, such a configuration may be adopted that combines the prediction generated by the first prediction means and the prediction generated by the second prediction means to calculate a final prediction result and use the final result to modify the prediction generated by the first prediction means.

When each of the first prediction means and the second prediction means uses a trained model, such a configuration using a two-stage prediction model may be adopted that the first prediction means uses a trained model that predicts the occurrence of a precursory stage of a disease, for example, the occurrence of a disease-prone state or a variation of a manifestation amount of a specific gene, and the second prediction means uses a trained model that predicts the contraction of the disease from the occurrence of the precursory stage of the disease.

### [Acquired Data]

The acquired data in the present invention is data including one or more selected from the group consisting of information on the diet, intestinal bacterial flora, body, living environment, diagnosis/medical checkup/examination, contracted diseases, and medical treatment of an animal.

### [Information on Diet]

The information on a diet of an animal is information relating to food that is ingested by the animal. Examples thereof include ingredients of food that is habitually eaten, the ingestion amount of food, and the number of times of ingestion. Examples of ingredients of food include concrete raw materials, and nutritional elements such as glucide, protein, lipid, and vitamins.

### [Information on Intestinal Bacterial Flora]

The information on intestinal bacterial flora is information relating to types and ratios of bacteria existing in the intestines of an animal. The intestinal bacterial flora can be grasped, for example, by acquiring a fecal sample of the animal and performing amplicon analysis (bacterial flora analysis) of 16SrRNA gene using NGS (next-generation sequencer). In addition, use may be made of a method of analyzing, with use of the next-generation sequencer, base sequence information of DNA and RNA of all living organisms included in the fecal sample obtained from the animal, thereby identifying the living organisms included in the sample. The information relating to intestinal bacterial flora may be an occupation rate (hit rate) of a specific bacterial family (or genus, order, class, or phylum) included in the intestinal bacterial flora, or the presence/absence of bacteria belonging to a specific bacterial family (or genus, order, class, or phylum). Examples of such a specific bacterial family include Alcaligenaceae, Bacteroidaceae, Bifidobacteriaceae, Clostridiaceae, Coprobacillaceae, Coriobacteriaceae, Enterobacteriaceae, Enterococcaceae, Erysipelotrichaceae, Fusobacteriaceae, Lachnospiraceae, Peptostreptococcaceae, Prevotellaceae, Ruminococcaceae, Veillonellaceae, Streptococcaceae, Campylobacteraceae, Desulfovibrionaceae, Flavobacteriaceae, Helicobacteraceae, Odoribacteraceae, Paraprevotellaceae, Peptococcaceae, Porphyromonadaceae, Succinivibrionaceae, Desulfovibrionaceae, Enterobacteriaceae, Lactobacillaceae, Turicibacteraceae, Comamonadaceae, Leuconostocaceae, Pseudomonadaceae, and Sphingobacteriaceae. It is preferable to use, as information on intestinal bacterial flora, the information relating to the occupation rate or presence/absence of one or more of these.

An example of the amplicon analysis (bacterial flora analysis) of the 16SrRNA gene using the NGS (next-generation sequencer) is concretely described. DNA is extracted from a sample such as feces using a DNA extraction reagent, and the 16SrRNA gene is amplified by PCR from the extracted DNA. Then, with respect to the amplified DNA fragment, the base sequence is exhaustively determined using the NGS, and a low-quality lead or chimera sequence is removed, and thereafter sequences are clustered and OTU (Operational Taxonomic Unit) analysis is performed. The OTU is a classification unit in operation for treating sequences having a similarity of a predetermined level or more (e.g., homology of 96 to 97% or more) like one bacterial type. Accordingly, it is considered that the number of OTUs represents the number of types of bacteria constituting the bacterial flora, and the number of leads belonging to the same OTU represents a relative existence amount of the type. A representative sequence is selected from the number of leads belonging to each OTU, and the family name and genus and species names can be identified by database searches. In this manner, the presence/absence and occupation rate of bacteria belonging to a specific family can be measured.

The data relating to the occupation rate is data relating to the occupation rates of respective bacteria included in the intestinal bacterial flora of the animal. The occupation rate is an existence ratio (detection ratio) of bacteria belonging to each bacterial family in the intestinal bacterial flora, and can be measured, for example, as a detection result “hit rate” in a publicly known metagenomic analysis method such as amplicon sequence using a sequencer such as the NGS. In the present invention, as the data relating to the occupation rate, use may be made of a numerical value of the occupation rate of the intestinal bacterial flora, or a label or score that is set based on the occupation rate. Besides, as regards the presence/absence of bacteria, a label or score that is set based on the presence/absence of bacteria may be used.

The occupation rate in the present invention is an occupation rate of each family of bacteria. The occupation rate of each family of bacteria is the occupation rate of the entire bacteria belonging to a certain family. Specifically, when the occupation rate for each family is calculated, occupation rates of bacterial species belonging to a certain family are totaled with respect to each bacterial species in the intestinal bacterial flora, and thus the occupation rate of this family can be calculated. The occupation rates may be totaled for each family by performing species-level or genus-level identification, or the occupation rate for the family may be calculated by performing family-level identification, without performing species-level or genus-level identification.

The label that is set based on the occupation rate is a label that is properly set in accordance with the magnitude of the numerical value of the occupation rate. For example, three-level labels, such as “large”, “middle” and “small”, or “many”, “medium” and “few”, can be set in accordance with the numerical value of the occupation rate. In addition, the number of levels of labels can freely be set, and, for example, labels of multiple levels, such as “0”, “1”, “2”, “3”, . . . , “20”, can be added.

In the case of using the label, the occupation rate in the intestinal bacterial flora is measured, and, before the numerical value is input to input means, a specific label is allocated from a preset correspondence table in accordance with the measured occupation rate, and this label can be input to reception means.

In addition, the label that is set based on the presence/absence of bacteria is a label that is properly set in accordance with the presence/absence of bacteria. For example, when bacteria are present, a label “1” can be added, and when bacteria are absent, a label “0” can be added.

The score that is set based on the occupation rate is a score that is properly set in accordance with the magnitude of the numerical value of the occupation rate of bacteria. For example, when the occupation rate is equal to or greater than a certain reference value, a score of “+1” is given, and when the occupation rate is less than the certain reference value, a score of “−1” is given.

In addition, the score that is set based on the presence/absence of bacteria is a score that is properly set in accordance with the presence/absence of bacteria. For example, with respect to a specific family, when a bacteria belonging to the family is present, a score of “+1” is given, and when the bacteria is absent, a score of “−1” is given.

Such a score may be calculated for each bacterial family and may be input to reception means. In addition, such a configuration may be adopted that the presence/absence and the occupation rate of bacteria belonging to the bacterial family are input to the reception means, and the score is calculated for each bacterial family, based on a score allocation standard that is preset based on the data on the presence/absence of bacteria and the occupation rate, which are input.

The prediction apparatus of the present invention may be configured such that the second prediction means totals scores that are calculated or input in regard to respective families, and performs a prediction of, or modifies a prediction of, the possibility of disease contraction, based on the obtained total score.

### [Information on the Body]

The information on the body of an animal is information relating to the external appearance and vital signs of the animal. Examples thereof include external information on the animal such as body length, body weight, coat of fur, and an alignment of teeth, as well as body temperature, pulsation, heart rate, respiration rate, blood pressure, and urinary and defecation frequency. These pieces of information may be in a classified form or in a scored form.

### [Information on the Living Environment]

The information on the living environment of an animal is information relating to the environment in which the animal is reared. Examples thereof include an address of a dwelling place where the animal is reared, the square footage of the dwelling house, whether it is an urban area, whether it is a detached house or an apartment, the number of floors of the dwelling place, and whether it is multiple animal breeding. Information about the owner is also included. These pieces of information may be in a classified form or in a scored form.

### [Information Relating to Diagnosis, Medical Checkup, and Examination]

The information on the diagnosis, medical checkup, and examination of an animal is information on the results of the diagnosis, medical checkup, and examination of the animal. Examples thereof include information relating to basic vital signs such as body temperature, pulsation, heart rate, respiration rate, and blood pressure, information on blood such as blood flow, uric acid level, and blood sugar level, information relating to excrements such as bloody stools and bloody urine, and information relating to non-invasive examinations of CT, MM and the like. These pieces of information may be in a classified form or in a scored form.

### [Information Relating to Contracted Disease]

The information on a contracted disease of an animal is information relating to a disease that the animal presently contracts or a disease that the animal contracted in the past. By acquiring the present or past information of contraction, the information can be utilized to predict disease contraction in the future. These pieces of information may be in a classified form or in a scored form.

### [Information Relating to Medical Treatment]

The information on medical treatment of an animal is information relating to the medical treatment the animal received and its prognosis. Examples thereof include medication information such as type of medicine, date/time, frequency, dosage, place, and a person providing medication (e.g., veterinarian), operation information such as type of operation (including radiotherapy), date/time, frequency, time of operation, place, and a surgeon, and prognosis information after medication or an operation. If a disease predicted by the first prediction means actually appeared and the disease was treated, the possibility of contracting the same disease lowers except for recurrent disease, and the second prediction means can modify the prediction by the first prediction means using the information on the medical treatment. These pieces of information may be in a classified form or in a scored form.

### (Reception Means)

The prediction apparatus of the present invention may include a reception means that receives an input of data. The reception method in the case of receiving an image may be any method such as scan, input or transmission of image data, and image take-in by photography on the spot.

### (Output of Prediction Result)

The output form of the prediction result by the second prediction means of the present invention is not particularly limited, and the prediction result can be output, for example, on the screen of a terminal such as a personal computer or a smartphone by displaying “There is a possibility of contracting diabetes within one year”, “There is a high possibility of contracting cancer within three years”, or “There is a * % (* is the number calculated) possibility of contracting cancer and consequently dying within five years.”

The prediction apparatus of the present invention may include a separate output means that receives a prediction result from the second prediction means and outputs the prediction result.

### (Proposing Means)

The prediction apparatus of the present invention may further include a proposing means that proposes a preventive plan for preventing the occurrence of a disease or disease-prone state, in accordance with the prediction result. For example, the proposing means can propose or recommend, in accordance with the prediction result generated by the second prediction means, food for avoiding a predicted disease risk, supplements including bacteria that make the disease less contractable, food with a low salt content and a low caloric value, low sugar food, a diet menu, and the like. The proposing means may include a trained model. It is preferable that the preventive plan includes a proposal for one or more changes in food, an exercise habits, lifestyle, living environment, clothing, and a primary care doctor.

In addition, it is possible to make or customize beverages, food, and supplements for preventing the occurrence of a disease or disease-prone state, in accordance with the prediction result that is output by the prediction apparatus or prediction method of the present invention. As services relating to the prediction, such modes are possible as a prediction by the prediction apparatus or prediction method of the present invention, the provision of the prediction result, the making or customization of beverages, food, and supplements corresponding to the prediction result, and the proposal and recommendation of the beverages, food, and supplements. In addition, it is also possible to further implement the prediction apparatus or prediction method of the present invention, for example, using only the second prediction means to present whether the possibility of contracting a disease has lowered. The above-described beverages, food and supplements include beverages for diet therapy, diet food, and additives for dietary supplements.

In this manner, by proposing, making, and customizing diets and foods corresponding to the prediction result, the reduction and avoidance of the disease risk are expected.

### (Request Reflection Means)

The prediction apparatus of the present invention may further include a request reflection means that limits a preventive plan proposed by the proposing means in accordance with a pet owner's request. In some cases, the preventive plan proposed by the proposing means includes proposals relating to a plurality of changes, and the load on the owner is too heavy. In such cases, the request reflection means can modify the preventive plan in accordance with the owner's request to provide a preventive plan with less load on the owner.

The modification of a preventive plan is a modification for reducing points of change in the preventive plan or for replacing the plan with another preventive plan. The modification for reducing points of change is a modification, for example, when food reduction and morning, afternoon, and evening exercise are proposed for an obese dog as a preventive plan, that limits the preventive plan to only food reduction and morning and evening exercise. The modification for replacing one preventive plan with another is a modification, for example, when food reduction is proposed for an obese dog as a preventive plan, that replaces the preventive plan with morning, afternoon, and evening exercise.

### [Medical Expense Calculation Means]

It is preferable that the prediction apparatus of the present invention includes medical expense calculation means that calculates a medical expense that the pet owner may possibly bear in the future, in accordance with the prediction modified by the second prediction means. The medical expense calculation means is composed of, for example, a program or software, and accesses a separately prepared list or database of medical expenses of various diseases, based on the prediction result generated by the second prediction means, and presents a rough estimate of the expense that the owner of the animal will be required to bear for the medical treatment of a disease. The list or database of medical expenses can be constructed by acquiring information relating to medical expenses by hearings from animal hospitals and policyholders of pet insurance.

### <Prediction System>

The prediction system of the present invention is configured such that the above-described prediction apparatus and terminals used by owners of animals are connected via a network. The owner of an animal can upload and input inborn data and acquired data of the animal to the prediction apparatus through a terminal such as a smartphone or a tablet. The inborn data and acquired data can also be uploaded to the prediction apparatus through a terminal by an analysis business dealer that deals with DNA sequence analysis and intestinal bacterial flora analysis upon receiving a request by the owner of the animal, or can also be uploaded to the prediction apparatus through terminals of animal hospitals.

### <Prediction Method>

A prediction method of the present invention includes a step of receiving inborn data including one or more selected from the group consisting of genetic information, pedigree information, and appearance information of an animal; a first prediction step of predicting the occurrence of a disease or disease-prone state in the future from the inborn data; a step of obtaining acquired data including one or more selected from the group consisting of information on eating habits, intestinal bacterial flora, body, living environment, diagnosis/medical checkup/examination, contracted disease, and medical treatment of the animal; and a second prediction step of correcting a prediction of a disease contraction possibility in the future predicted from the inborn data, based on the acquired data.

The method for prediction and the configuration therefor are the same as described in connection with the above-described prediction apparatus.

### Embodiments

Examples of embodiments of the prediction apparatus and the prediction system of the present invention are described with reference to the drawings.

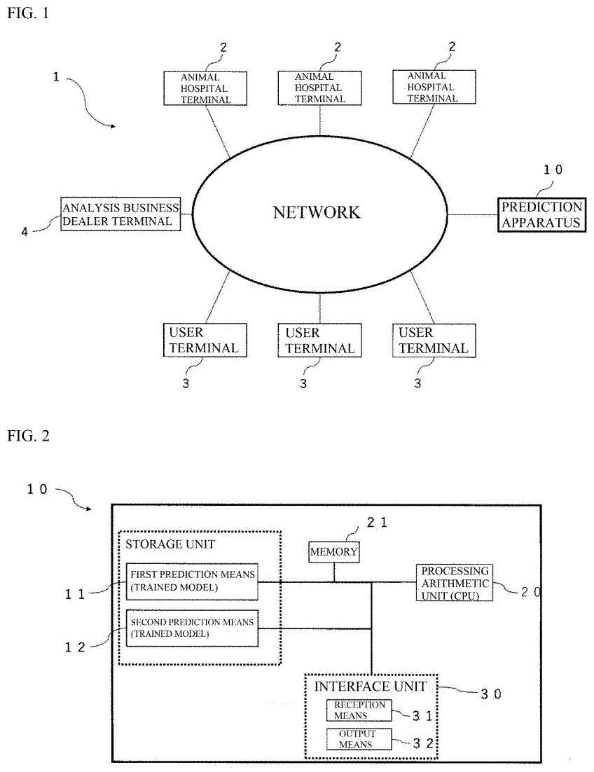

FIG. 1 illustrates an example of a prediction system 1 of the present invention. In the prediction system 1, a prediction apparatus 10 of the present invention is connected to animal hospital terminals 2, user terminals 3, and an analysis business dealer terminal 4 via a network.

In FIG. 1, the user terminal 3 is a terminal used by a person (user) who wishes to utilize the prediction apparatus. Examples of the terminal 3 include a personal computer, a smartphone, and a tablet terminal. The terminal 3 is configured to include a processing unit such as a CPU, a memory/storage unit such as a hard disk, ROM or RAM, a display unit such as a liquid crystal panel, an input unit such as a mouse, a keyboard or a touch panel, and a communication unit such as a network adapter.

The user accesses the prediction apparatus 10 from the terminal 3 through the network, and inputs and transmits inborn data and acquired data of an animal that is an object, and, where necessary, information such as a facial image (photograph), and the name, kind, breed, age and medical history of the animal.

The user can receive a prediction result by accessing the prediction apparatus 10 from the terminal 3.

The prediction system of the present invention can include the animal hospital terminal 2 that is installed in an animal hospital. The animal hospital terminal 2 is connected to the prediction apparatus 10 through the network. At a time such as when diagnosing the target animal, the animal hospital can upload, on behalf of the user, acquired data including one or more selected from the group consisting of information on the eating habits, intestinal bacterial flora, body, living environment, diagnosis/medical checkup/examination, contracted disease, and medical treatment of the animal.

The prediction system of the present invention can include the analysis business dealer terminal 4. The analysis business dealer is a business dealer that performs analysis of the DNA, intestinal bacterial flora and the like of the animal, upon receiving a request from the user. The analysis business dealer can upload the genetic information of the animal and the information relating to the intestinal bacterial flora to the prediction apparatus through the analysis business dealer terminal 4, in place of presenting the analysis result to the user, or along with presenting the analysis result to the user.

FIG. 2 illustrates an example of the prediction apparatus 10 of the present invention. In the present embodiment, the prediction apparatus 10 is composed of a computer but may be any apparatus as far as the apparatus includes the functions relating to the present invention.

A storage unit is composed of, for example, ROM, RAM, or a hard disk. The storage unit stores an information processing program for causing the components of the prediction apparatus to operate, and stores, in particular, software for a first prediction means 11 and a second prediction means 12.

A CPU 20 functions as the first prediction means or the second prediction means by executing the program/software relating to the first prediction means or the program/software relating to the second prediction means.

As described above, the user or the business dealer that measured the intestinal bacterial flora inputs the inborn data of the target animal to the first prediction means 11, and the first prediction means 11 outputs a prediction as to whether the animal contracts a predetermined disease or enters a disease-prone state within a predetermined period (e.g., within one year, three years, five years, or a lifetime) or as to what % of the possibility thereof is. As the prediction means, a trained model may be used. Such a trained model is configured to include, for example, XGBoost, CatBoost, LightGBM, or deep or convolutional neural networks.

As described above, the user or the business dealer that measured the intestinal bacterial flora inputs the acquired data of the target animal to the second prediction means 12, and the second prediction means 12 modifies the prediction output by the first prediction means. As the prediction means, a trained model may be used. Such a trained model is configured to include, for example, XGBoost, CatBoost, LightGBM, or deep or convolutional neural networks.

In the present embodiment, such a mode was described that the first prediction means, the second prediction means, and the reception means are accommodated in the prediction apparatus, and are connected to the terminals of the users by the connection means such as the internet or LAN. However, the present invention is not limited to this, and such a mode may be adopted that the first prediction means, second prediction means, reception means, and interface unit are accommodated in one server or apparatus, or that the terminal used by the user is not separately needed.

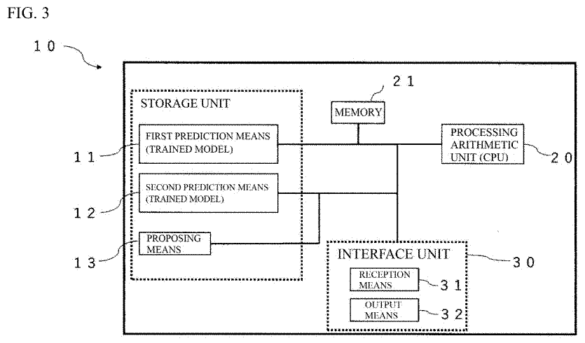

As illustrated in FIG. 3, the prediction apparatus of the present invention may include proposing means 13. The proposing means 13 is a program or software for proposing a method for avoiding such a situation as to contract a disease or enter a disease-prone state in accordance with the prediction output by the first prediction means 11 or second prediction means 12. For example, the proposing means calls and outputs, in accordance with the prediction result, information relating to recipes for food for improving various types of diseases or disease-prone states, composition recipes of supplements, and hospitals having reputations for treating such diseases stored in a storage unit or a separately prepared database. Concretely, when the occurrence of diabetes is predicted, a recipe of low levels of sugar food, a supplement assisting secretion of insulin, or a list of hospitals that are known to be excellent in the treatment of diabetes is presented, or the reduction in the amount of food or the increase in the amount of exercise is proposed.

The processing arithmetic unit (CPU) 20 executes the prediction of the occurrence of a disease or disease-prone state using a program or software, stored in the storage unit, for the first prediction means 11 and the second prediction means 12.

An interface unit (communication unit) 30 includes reception means 31 and output means 32, receives inborn and acquired data of an animal and other information where necessary from the user's terminal, and outputs and transmits to the user's terminal a prediction result relating to the occurrence of a disease or disease-prone state.

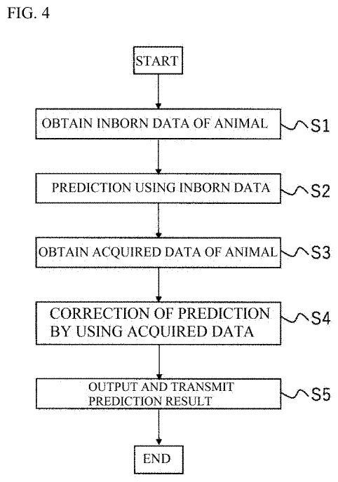

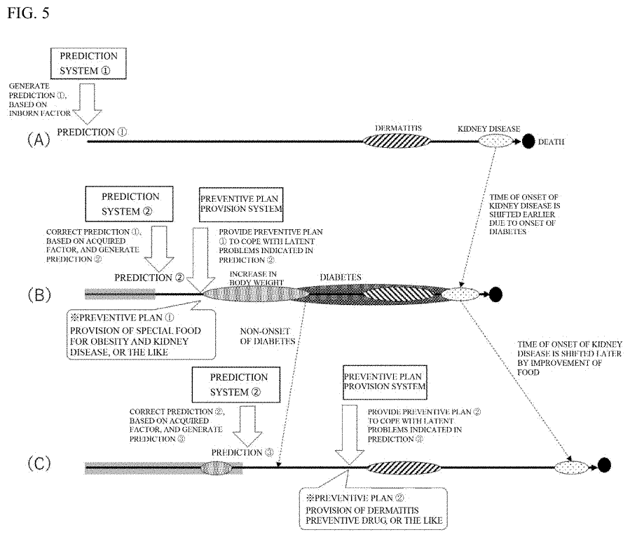

An example of the prediction of the occurrence of a disease or disease-prone state, which is executed by the prediction apparatus of the present invention, is described with reference to FIG. 4.

For the purpose of convenience of illustration, this embodiment is described including also the acquisition of a specimen from the animal and the data acquisition with respect to the intestinal bacterial flora. The user samples a specimen from the animal using a DNA sampling kit or the like, sends the specimen to the analysis business dealer, acquires inborn data of the animal such as genetic information, and inputs the inborn data to the prediction apparatus of the present invention (step S1). The prediction apparatus of the present invention predicts the occurrence of a disease or disease-prone state from the inborn data (step S2). Next, the user samples a fecal sample of the animal using a feces sampling kit or the like and sends the fecal sample to the analysis business dealer. The analysis business dealer analyzes the intestinal bacterial flora of the animal using the sample. Then, the user or analysis business dealer inputs to the prediction apparatus the acquired data such as the information relating to the intestinal bacterial flora (step S3). The prediction apparatus of the present invention modifies, based on the acquired data, the prediction of the occurrence of the disease or disease-prone state (step S4). The prediction apparatus outputs and transmits the prediction to the terminal, and the prediction result is displayed on the terminal (step S5).

### Other Embodiments

Examples of other embodiments of the prediction apparatus and prediction system of the present invention are described with reference to FIG. 5.

FIG. 5A is a schematic diagram in a case where, in the prediction apparatus of the present invention, the first prediction means predicts the occurrence of a disease in the future using inborn data. In FIG. 5A, the right-pointing arrow indicates the passage of time toward the future. In FIG. 5A, the contraction of dermatitis and the subsequent contraction of kidney disease are predicted. In particular, in FIG. 5A, since death comes after the contraction of the kidney disease, the possibility of dying due to the kidney disease is suggested. This prediction is derived when the presence of causal genes of dermatitis and kidney disease is included in the input genetic information of the animal that is the object. The time of the onset can be predicted by taking into account, for example, the pedigree information, breed information, and the like, in addition to the genetic information.

Next, FIG. 5B is a schematic diagram in a case where a prediction (2) was generated by modifying the prediction (prediction (1)) derived by the first prediction means based on the acquired data. The second prediction means predicts the occurrence of a disease or disease-prone state using the acquired data such as information relating to the body weight, eating habits, and intestinal bacterial flora, and, as a result (prediction (2)) of the modification of the prediction (1), a prediction of an increase in body weight (obesity) and subsequent contraction of diabetes in an earlier stage than the contraction of dermatitis was derived. In addition, the prediction of the time of the onset of kidney disease in the prediction (2) is shifted to an earlier time than in the prediction (1).

To be more specific, the second prediction means derives the prediction (2) by correcting the prediction (1), based on a model in which “if the body weight and the body fat ratio increase at a fixed rate, obesity occurs in the year of xx, diabetes is contracted in the year of ∘∘, and the time of contraction of a disease (e.g., kidney disease), the contraction of which becomes earlier due to diabetes, becomes earlier by ΔΔ year(s).

In the embodiment illustrated in FIG. 5B, the proposing means proposes special food for the obesity and kidney disease to cope with the latent problems of the increase in body weight and the kidney disease indicated in the prediction (2) (preventive plan (1)).

FIG. 5C illustrates an example in which the prediction result was modified once again by the second prediction means, in a case where diabetes does not occur even at the predicted time of occurrence, as a result of the execution of the preventive plan (1). In FIG. 5C, it is indicated that the increase in body weight is suppressed by executing the preventive plan (1). In addition, diabetes did not develop even at the time when the occurrence was predicted. If the prediction is modified once again by the second prediction means using the acquired data that the increase in body weight or diabetes did not occur (prediction (3)), a lower arrow is obtained. As a result of the re-modification of the prediction by the second prediction means, the time of the prediction of contraction of the kidney disease is shifted later, and accordingly, the time of the prediction of death is shifted later, and the predicted lifetime is increased. The reason why the predicted time of the onset of the kidney disease is shifted later is that the special food for the kidney disease was provided by the proposal of the preventive plan (1). On the other hand, in the prediction (2), as regards the onset of dermatitis, no particular modification is made.

In FIG. 5C, in order to cope with the prediction of dermatitis, the proposing means proposes the provision of a dermatitis preventive drug (preventive plan (2)).

Another embodiment is illustrated.

As a result of a genetic test in regard to a newborn puppy (collie), such inborn data on genetic information that the puppy has a mutation of an MDR1 gene was obtained, and the inborn data relating to the genetic information, together with the information relating to the breed, age, and pedigree of the dog, is input to the prediction apparatus of the present invention. In connection with this dog, the presence of a disease-related genetic mutation, other than the mutation of the MDR1 gene, is not confirmed. In general, if the mutation of the MDR1 gene is present, it is known that toxicosis tends to develop when ivermectin, which is a filariasis preventive drug, is administered (ivermectin sensitivity). On the other hand, toxicosis is not basically developed if ivermectin is not administered, and whether or not to administer ivermectin depends on the owner's intent, which is an acquired element. Thus, a prediction of not contracting a disease or the like is derived in the first prediction means (prediction (4)).

In addition, such acquired data on a living environment in which the dog is reared outdoors is input to the prediction apparatus of the present invention. The prediction apparatus of the present invention modifies the prediction (4) by the second prediction means using a trained model which takes into account that the probability of the onset of filariasis is high in dogs reared outdoors, and that owners of a fixed ratio perform filariasis vaccination, and such a prediction is derived that the dog develops ataxia due to filariasis at one year old (prediction (5)). Since the trained model generated by machine learning is used, the reason why the age of one year was indicated is not clear, but a decrease in the attentiveness of the owner, the speed of growth, and the like are considered to be factors.

Note that in the present embodiment, since the first prediction means and the second prediction means are executed as one piece at the same timing, the owner receives only the prediction (5), without receiving the prediction (4).

Next, in connection with the prediction (5), the proposing means proposes an injection of moxidectin which is a filariasis preventive drug at a nearby animal hospital A (preventive plan (3)). From the viewpoint of the program, such another proposal can be examined that a minimum necessary dose of ivermectin, which does not develop ivermectin toxicosis even if there is the MDR1 genetic mutation, is periodically administered by a veterinarian, this proposal is excluded since the load on the owner is heavy and the disease risk is high.

Moreover, in the present embodiment, since the owner makes such a request that not a nearby animal hospital, but an animal hospital near a park where the owner visits frequently is preferable, the request reflection means proposes a moxidectin injection in an animal hospital B near the park (preventive plan (4)). The owner is also provided with information that a veterinarian familiar with ivermectin toxicosis is on the register in the animal hospital B and the diagnosis results are good.

1. A prediction apparatus comprising:
a first prediction means for predicting the occurrence of future disease or disease-prone state in an animal based on inborn data including one or more selected from the group consisting of genetic, pedigree, and appearance information of the animal; and
a second prediction means for modifying, using a trained model, a prediction generated by the first prediction means based on acquired data including one or more selected from the group consisting of information on diet, bacterial flora, body, living environment, diagnosis/medical checkup/examination, contracted disease, and medical treatment of the animal.

2. The prediction apparatus according to claim 1, further comprising a proposing means for proposing a preventive plan for preventing the occurrence of the disease or disease-prone state in accordance with the prediction modified by the second prediction means.

3. The prediction apparatus according to claim 2, wherein the preventive plan includes a proposal for one or more changes of food, exercise habits, lifestyle, living environment, clothing, and primary care doctor.

4. The prediction apparatus according to claim 1, wherein the modification by the second prediction means is a modification that delays the timing of occurrence or lowers the probability of occurrence of the future disease or disease-prone state predicted by the first prediction means.

5. The prediction apparatus according to claim 1, wherein the disease-prone state is one or more selected from the group consisting of obesity, low body weight, high blood pressure, low blood pressure, hyperglycemia, and hypoglycemia.

6. The prediction apparatus according to claim 1, further comprising a medical expense calculation means for calculating a medical expense that an owner of an animal possibly bears in the future in accordance with the prediction modified by the second prediction means.

7. The prediction apparatus according to claim 1, wherein the disease is a hereditary disease.

8. The prediction apparatus according to claim 1, wherein the disease is a lifestyle-related disease.

9. The prediction apparatus according to claim 1, wherein the genetic information is information on a sequence or mutation of one or more selected from the group consisting of a cancer-related gene, a progressive retinal atrophy (PRA)-related gene, a hereditary cataract-related gene, a collie eye anomaly (CEA)-related gene, a von Willebrand's disease (vWD)-related gene, an MDR1 gene, a copper-storage hepatopathy-related gene, a cystinuria-related gene, an osteogenesis imperfecta-related gene, an X-chromosome-linked muscular dystrophy-related gene, a voltage-gated chloride channel gene, an orexin-related gene, a severe combined immunodeficiency-related gene, a leukocyte adhesion deficiency (CLAD)-related gene, a cyclic neutropenia (gray collie syndrome)-related gene, a phosphofructokinase deficiency-related gene, a pyruvate kinase deficiency-related gene, and a lysosomal storage disease-related gene.

10. The prediction apparatus according to claim 1, wherein the second prediction means is capable of further modifying the modified prediction by the first prediction means using acquired data other than the acquired data once used.

11. The prediction apparatus according to claim 2, further comprising a request reflection means for modifying the preventive plan proposed by the proposing means in accordance with a request of an owner of an animal.

12. A prediction system wherein the prediction apparatus according to claim 1 and a terminal used by an owner of an animal are connected via a network.

13. A prediction method comprising:
a step of a computer receiving inborn data including one or more selected from the group consisting of genetic, pedigree, and appearance information of an animal;
a first prediction step of the computer predicting the occurrence of a future disease or disease-prone state in the animal based on the inborn data;
a step of the computer obtaining acquired data including one or more selected from the group consisting of information on diet, intestinal bacterial flora, body, living environment, diagnosis/medical checkup/examination, contracted disease, and medical treatment of the animal; and
a second prediction step of the computer modifying, using a trained model, a prediction on the occurrence of the future disease or disease-prone state predicted from the inborn data based on the acquired data.

14. The prediction apparatus according to claim 2, wherein the modification by the second prediction means is a modification that delays the timing of occurrence or lowers the probability of occurrence of the future disease or disease-prone state predicted by the first prediction means.

15. The prediction apparatus according to claim 3, wherein the modification by the second prediction means is a modification that delays the timing of occurrence or lowers the probability of occurrence of the future disease or disease-prone state predicted by the first prediction means.

16. The prediction apparatus according to claim 2, wherein the disease-prone state is one or more selected from the group consisting of obesity, low body weight, high blood pressure, low blood pressure, hyperglycemia, and hypoglycemia.

17. The prediction apparatus according to claim 3, wherein the disease-prone state is one or more selected from the group consisting of obesity, low body weight, high blood pressure, low blood pressure, hyperglycemia, and hypoglycemia.

18. The prediction apparatus according to claim 4, wherein the disease-prone state is one or more selected from the group consisting of obesity, low body weight, high blood pressure, low blood pressure, hyperglycemia, and hypoglycemia.

19. The prediction apparatus according to claim 2, further comprising a medical expense calculation means for calculating a medical expense that an owner of an animal possibly bears in the future in accordance with the prediction modified by the second prediction means.

20. The prediction apparatus according to claim 3, further comprising a medical expense calculation means for calculating a medical expense that an owner of an animal possibly bears in the future in accordance with the prediction modified by the second prediction means.
